SEQUENCE LISTING (15 sequences)

SEQ ID NO: 1 - DNA, 564 bp, Canis familiaris
atggtgcgcacgctgaactgcatcgtcgccgtgtcccagaacatgggcatcggcaggaacggggacctgccctggcccccgctcaggaatgaattcaagtatttccaaagaatgaccacgaactcctccgtggcaaggtaaacagaatttggtgattatgggtaggaagacatggttctctattcttgagaagaatcgaccgttaaaggacagaattaatatagttctcagcagagacctcaaggaacctccacaaggagctcattttcttgccaaaagtcggatgatgctttaaaacttactgagcaaccagaattagcaaataaagtggacatggtttggatagtgggaggcagttctgtttataaggaagccatgaacaaaccaggccatcttagactatttgtgacaaggattatgcatgaatttgaaagtgacacgtttttcccagaaattgatttggagaaatataaacttctgccagaatacccaggtgtgctttctgatgtccaggaggagaaaggcattaagtacaaatttgaagtatatgagaagaacgattaa

SEQ ID NO: 2 - protein, 188 aa, monkey
MDIAVNCIVAVDEQLGIGKNGTMPWPYLRNEMMYFQKMTSTPSVVGEKNVVIMGKRTWFSIPEKKRPLVNRINIILSRELREPPHGAHFLARTLDDAFNFYRQYKLKEQLNTVWVIGGKSVYESVLNYKCPLKLYITRIMEFSDCDVFFPSINFTEYLMLSELPGKDTNFEENGIKYKFQVYEKNFNK

SEQ ID NO: 3 - protein, 187 aa, rat
MVRPLNCIVAVSQNMGIGKNGLDPWPLLRNEFKYFQRMTTTSSVEGKQNLVIMGRKTWFSIPEKNRPLKDRINIVLSRELKEPPQGAHFLAKSLDDALKLIEQPELASKVDMVWVVGGSSVYQEAMNQPGHLRLFVTRIMQEFESDTFFPEIDLEKYKLLPEYPGVLSEIQEEKGIKYKFEVYEKKD

SEQ ID NO: 4 - protein, 187 aa, mouse
MVRPLNCIVAVSQNMGIGKNGLDPWPPLRNEFKYFQRMTTTSSVEGKQNLVIMGRKTWFSIPEKNRPLKDRINIVLSRELKEPPRGAHFLAKSLDDALRLIEQPELASKVDMVWIVGGSSVYQEAMNQPGHLRLFVTRIMQEFESDTFFPEIDLGKYKLLPEYPGVLSEVQEEKGIKYKFEVYEKKD

SEQ ID NO: 5 - protein, 186 aa, human
VGSLNCIVAVSQNMGIGKNGDLPWPFLRNEFRYFQRMTTTSSVEGKQNLVIMGKKTWFSIPEKNRPLKGRINLVLSRELKEPPQGAHFLSRSLDDALKLTEQPELANKVDMVWIVGGSSVYKEAMNHPGHLKLFVTRIMQDFESDTFFPEIDLEKYKLLPEYPGVLSDVQEEKGIKYKFEVYEKND

SEQ ID NO: 6 - protein, 376 aa, herpes simplex virus
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHVGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWWEDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 7 - protein, 427 aa, Escherichia coli
MSNNALQTIINARLPGEEGLWQIHLQDGKISAIDAQSGVMPITENSLDAEQGLVIPPFVEPHIHLDTTQTAGQPNWNQSGTLFEGIERWAERKALLTHDDVKQRAWQTLKWQIANGIQHVRTHVDVSDATLTALKAMLEVKQEVAPWIDLQIVAFPQEGILSYPNGEALLEEALRLGADVVGAIPHFEFTREYGVESLHKTFALAQKYDRLIDVHCDEIDDEQSRFVETVAALAHHEGMGARVTASHTTAMHSYNGAYTSRLFRLLKMSGINFVANPLVNIHLQGRFDTYPKRRGITRVKEMLESGINVCFGHDDVFDPWYPLGTANMLQVLHMGLHVCQLMGYGQINDGLNLITHHSARTLNLQDYGIAAGNSANLIILPAENGFDALRRQVPVRYSVRGGKVIASTQPAQTTVYLEQPEAIDYKR

SEQ ID NO: 8 - protein, 192 aa, mouse
MDGSGEQLGSGGPTSSEQIMKTGAFLLQGFIQDRAGRMAGETPELTLEQPPQDASTKKLSECLRRIGDELDSNMELQRMIADVDTDSPREVFFRVAADMFADGNFNWGRVVALFYFASKLVLKALCTKVPELIRTIMGWTLDFLRERLLVWIQDQGGWEGLLSYFGTPTWQTVTIFVAGVLTASLTIWKKMG

SEQ ID NO: 9 - DNA, 1954 bp, rat
aaaaaaaaaaaagatacaaatagttaaagacagaaagaagagctgccctcggtggcacacacctttactccctgcacaggagcagaggcagcagatggcagatctctatgttccaggccagcttggtctacagactgagatccaggagagccagggctacacagagaagctatttcaaataacaaacaaacactcagggtgctttcagggcttttcaacagaggactcaagccaagagactttctgactcccagaaactctactcttccctgctcaacacttcccccggtcttaggtctcagccaccctcagaagactcatagactcctactagaatattccagaatctgtcctgacctgctgctacttcacattgtccctctttcttctcttcttcttttctcctcctccttctcttcctattcttttttccttccttccttccttccttccttccttccttccttccttccttccttccttccttccttccttcctttgagacagggtttctctaagtagccatggctgtcgtggatctggctttgtagtccagattgcccttaaactcagagatctaccttcctgcatcctgagagctgggattaaaagtgtacacaactaccacccgactcttctcctcctcctcctcctccttttcttgaggctaaaggtctctcttggtatcctatgctggcttcatagggcttccatgttccgggattacaggtatgggccacaataccagggtctccttgctcctctcatcttcctctttgttttatgctttgagtgatggtctcagtatctagtgtaattgacctgtctcttgctatgtagacttgttgtgttttataaaaaagaaatagaggcaggatatgttttggtagaacatggtatggtgaggtgggaggggtgccccggcaggcccatactgaatccccctgaagtaccagccatgtagtatgtagtgccagatgtagtaccacgtagtgccaaatgtagtatggaatagagtttatttagggcatgggaagtggagttgggaagggaggaggggggaaggggagagaaaaggaggggagaggagatggccaggaactcatggaatgggctggaggggtgcggggaagaaggggcagaaagtaagaggataagagagtatgagagcttggacagggcagatagccccttttatagtaggtcaggcatacttgtctgttgccaggtaactgtgtgtgtgtgggggagactagaaggaatgctaaatagcctgggttgatcttaatttcagagacatctgcttgtttgtagctctgaaggctggtattaaaagacattggagccaccacacacagcctgtttttgttttttgaaatagtttcttagtagtgcagcttggactcaaactatgcaatccaggcttgcctcaaactccttagcaatcttcttgcttcactctatcagacctatggaccatcactctggtcaagtgttttgttttgttatgttttttttccagattggggcaatctcgctacagtcctggctggctttgagtttgtggcaggcaatattccgtcctgcctcagtctcccaatgctgggatgagaagcatatcctaggcaggctttgaacttgcggcaattctgctctaacctccttagtactctctaccatgaatctaatgaaggagaaataatagaaaacaaaaacaaaaacaaaaacaaaaaaccaaccctgggaaccagtccaggttcgggtagcgccgcgcatgcgtgaatttaatggcagaggccccgccccgaaagcgcgcgacgatcacgtgactagttctgcggggcggaggccatgttgcggggcacccacgtgagggccgcacgtctgcggggagtcacgtgaccggggcgcgccgcagccgccggggcgcacccggcgagaggcagcggcagtg

SEQ ID NO: 10 - protein, 192 aa, human
MDGSGEQPRGGGPTSSEQIMKTGALLLQGFIQDRAGRMGGEAPELALDPVPQDASTKKLSECLKRIGDELDSNMELQRMIAAVDTDSPREVFFRVAADMFSDGNFNWGRVVALFYFASKLVLKALCTKVPELIRTIMGWTLDFLRERLLGWIQDQGGWDGLLSYFGTPTWQTVTIFVAGVLTASLTIWKKMG

SEQ ID NO: 11 - protein, 281 aa, human
MQQPFNYPYPQIYWVDSSASSPWAPPGTVLPCPTSVPRRPGQRRPPPPPPPPPLPPPPPPPPLPPLPLPPLKKRGNHSTGLCLLVMFFMVLVALVGLGLGMFQLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 12 - protein, 281 aa, chicken anemia virus; identical to SEQ ID NO: 11

SEQ ID NO: 13 - protein, 237 aa, Aequorea victoria
SKGEELFTGVVPILVELDGDVNGQKFSVSGEGEGDATYGKLTLKFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMADKPKNGIKVNFKIRHNIKDGSVQLADHYQQNTPIGDGPVLLPDNHYLSTQSALSKDPNEKRDHMILLEFVTAAGITHGMDELYK

SEQ ID NO: 14 - protein, 376 aa, herpes virus
MASYPCHQHASAFDQAARSRGHSNRRTALRPRRQQEATEVRLEQKMPTLLRVYIDGPHGMGKTTTTQLLVALGSRDDIVYVPDPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 15 - DNA, 4151 bp, Artificial
  Cloning vector pEGFP-1, complete sequence, enhanced green fluorescent protein (egfp) and neomycin phosphotransferase genes
tagttattactagcgctaccggactcagatctcgagctcaagcttcgaattctgcagtcgacggtaccgcgggcccgggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaggcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgccatgcat

(19) United States
(12) Patent Application Publication
(10) Pub. No.: US 2004/0053836 A1
(43) Pub. Date: Mar. 18, 2004

(54) Method for modulating the production of a selected protein in vivo

(76) Inventors: Philipp Mayer-Kuckuk, New York, NY (US); Debabrata Banerjee, Bellerose, NY (US); Joseph Bertino, Branfrod, CT (US)

Correspondence Address: OPPEDAHL AND LARSON LLP, P O BOX 5068, DILLON, CO, 80435-5068, US

(21) Appl. No.: 10/421,285

(22) Filed: Apr. 22, 2003

Related U.S. Application Data
(60) Provisional application No. 60/375,250, filed on Apr. 22, 2002.

(51) Int. Cl. A61K 48/00; A61K 38/17; C12P 21/02; C12N 5/06; C07K 14/705

(52) U.S. Cl. 514/12; 514/44; 530/350; 536/23.5; 435/69.7; 435/320.1; 435/325

(57) ABSTRACT

A method is provided for use in producing a selected protein in mammalian cells, and to cDNA molecules useful in the method, and fusion proteins produced from expression of the cDNA. In the method, cDNA encoding a fusion protein that includes a mammalian DHFR and the selected protein is introduced into mammalian cells such that it is expressed. The naturally occurring repression of DHFR translation is overcome by treatment of the cells with a folate or antifolate or similar composition. The relief from this repression extends to the selected protein which is the second part of the expressed fusion, such that the treatment results in controllable and enhanced production of the selected protein.

This application claims the benefit of U.S. Provisional Application Serial No. 60/375,250 filed Apr. 22, 2002, which application is incorporated herein by reference in its entirety.

The invention disclosed in this application was supported by grants CA-08010 and CA-86438-02 from the National Cancer Institute. The United States Government may have certain rights in this invention.

### BACKGROUND OF THE INVENTION

This application relates to a method for modulating the production of a selected protein in vivo by controllable translational upregulation. The method makes use of cDNA encoding fusion proteins which contain a gene encoding dihydrofolate reductase (DHFR) and a second gene encoding the selected protein.

In mammals, DHFR levels in vivo are regulated in part because DHFR inhibits its own translation by binding to its cognate RNA within the coding region. This repression of protein synthesis is relieved when antifolates such as methotrexate (MTX) or trimetrexate (TMTX) are administered, leading to renewed protein synthesis.

### SUMMARY OF THE INVENTION

The present invention provides a method for use in producing a selected protein in mammalian cells, to cDNA molecules useful in the method, and to fusion proteins produced from expression of the cDNA. In accordance with the invention, cDNA molecules encoding a fusion protein that comprises mammalian DHFR and the selected protein is introduced into mammalian cells such that it is expressed. The naturally occurring repression of DHFR translation is overcome by treatment of the cells with a folate or antifolate or similar composition. The relief from this repression extends to the selected protein which is the second part of the expressed fusion, such that the treatment results in controllable and enhanced production of the selected protein.

### BRIEF DESCRIPTION OF THE DRAWINGS

FIG. 1 shows an exemplary fusion cDNA in accordance with the invention;

FIG. 2 shows Western blot results demonstrating increased expression of a DHFR HSV-TK fusion protein in response to antifolate treatment;

FIG. 3 shows increase in sensitivity to GCV by cells transduced with DHFR-HSV-TK fusion protein;

FIG. 4 shows increased accumulation of ¹⁴C-FIAU in cells transduced with DHFR-HSV-TK fusion protein;

FIG. 4 shows that ³H-thymidine incorporation is comparable in cells transduced with DHFR-HSV-TK fusion protein and untransduced cells;

FIG. 6 shows antitumor response of transduced tumor cells;

FIGS. 7A-D show GCV sensitivity and TMTX resistance of parental, transduced and TMTX-exposed colon cancer cells; and

FIG. 8 shows fusion cDNAs containing the EGFP gene.

### DETAILED DESCRIPTION OF THE INVENTION

The present invention provides a method for use in producing a selected protein in mammalian cells, and to cDNA molecules useful in the method. In accordance with the invention, cDNA molecules encoding a fusion protein that comprises mammalian DHFR and the selected protein is introduced into mammalian cells such that it is expressed. The naturally occurring repression of DHFR translation is overcome by treatment of the cells with a therapeutic agent that causes DHFR to release from its cognate RNA, for example a folate or antifolate. The relief from this repression extends to the selected protein which is the second part of the expressed fusion, such that the antifolate treatment results in controllable and enhanced production of the selected protein.

Thus, in a first aspect, the present invention provides a cDNA molecule encoding a fusion protein that comprises mammalian DHFR and a therapeutic protein. The sequences of several types of mammalian DHFR are known in the art, including dog, monkey, rat and mouse (Seq. ID. Nos. 1-4, respectively). The wild-type sequence of human DHFR is also known. (Seq. ID. No. 5) Masters et al., Gene 21: 59-63 (1983). The mammalian DHFR included in the cDNA molecule may be a wild-type sequence, or it may be a mutant form in which one or more amino acids are changed to alter resistance to antifolates such as methotrexate.

In one set of mutations, the mutations is such that the mutant DHFR has increased resistance to methotrexate. Methotrexate can inhibit the activity of such mutants to some extent, and remains effective to remove the DHFR protein from the RNA, leading to a resumption of translation. In addition, the mutant DHFR can confer resistance to transfected cells, allowing for usage of higher doses of methotrexate without killing the cells, and for selection of transformed cells. In particular, the DHFR encoded by the cDNA may be a mutant form of human DHFR that differs from wild-type human DHFR as a result of one or more mutations, including at least one mutation at an amino acid corresponding to amino acid 15, 22, 31 or 34 of the wild-type sequence. As used in the specification and claims of this application, the term “corresponding” refers to an amino acid residue that occupies the same function position (e.g., a position within the active site) even though the amino acid number may be different as a result of insertions or delations elsewhere in the protein.

International Patent Publication No. WO94/24277, which is incorporated herein by reference, discloses mutant forms of human DHFR which have increased resistance to inhibition by antifolates used in therapy including MTX. The specific mutants disclosed differ from wild-type human DHFR as a result of a single mutation occurring at amino acid 15, 31 or 34. Mutations at amino acid 22 of human DHFR have also been shown to reduce the sensitivity of the enzyme to antifolate inhibition. Ercikan et al., in Chemistry and Biology of Pteridines and Folates, J. E. Ayling, ed., Plenum Press (1993). In these mutants, the amino acids isoleucine, methionine, phenylalanine and tyrosine are substituted for the leucine of the wild-type enzyme. A particular mutant form of human DHFR encoded by the cDNA differs from wild-type human DHFR as a result of a set of mutations comprising a mutation at the amino acid corresponding to amino acid 22 in the wild-type sequence and a mutation at the amino acid corresponding to amino acid 31 in the wild-type sequence, for example Ser31Tyr22, Ser31Phe22, Gly31Tyr22, Gly31Phe22, Ala31Tyr22 and Ala31Phe22 mutants as described in International Patent Publication WO97/33988, which is incorporated herein by reference.

The cDNA molecule of the invention encodes a fusion protein that also comprises a therapeutic protein or peptide. This therapeutic protein, may be any protein or peptide which is desirably produced in a treated subject, particularly where it may be desirable to be able to control the expression levels of the therapeutic protein.

One class of proteins which may be incorporated as the therapeutic protein in the invention are protein products which can be used to enhance the toxicity of an administered drug. The genes encoding these proteins are sometimes called “suicide genes.” For example, herpes simplex virus thymidine kinase (HSV-TK) which can be used in gancyclovir (GCV) therapy to convert administered GCV into a cytotoxic derivative. HSV-TK can also convert acyclovir or 1-(2-deoxy-2-fluoro-.beta.-D-arabinofuranosyl)-5-iodouracil (FIAU) into toxic substances. Any type of HSV-TK which is effective to catalyze at least one of these conversions may be used in the present invention. By way of non-limiting example, the sequence of the gene coding for the herpes simplex virus type 1 thymidine kinase enzyme has been described in the literature (see, McKnight et al., Nucl. Acids Res. 8, 5949-5964 (1980), GenBank Accession No. J02224, Seq. ID No. 6). Natural variants of HSV-TK exist, leading to proteins having a comparable enzyme activity with respect to thymidine, or ganciclovir (M. Michael et al., 1995 Biochem. Biophys. Res. Commun 209, p. 966). Similarly, derivatives have been described which were obtained by directed mutagenesis at the binding site of the enzyme with the substrate. For example, U.S. Pat. Nos. 6,245,543 and 6,207,150, which are incorporated herein by reference describe mutant forms of HSV-TK.

Another gene of this type is the codA gene of Escherichia coli encoding cytosine deaminase which can be used for the selective elimination of unwanted human cells. (See, Austin, E. A. et al., Mol. Pharmacol. 43 (3), 380-387 (1993), GenBank Accession No. S56903, Seq. ID No. 7) Cytosine dearninase is the first enzyme of the only metabolic pathway by which exogeneous cytosine or endogeneous cytosine from pyrimidine nucleotide breakdown is utilized by way of hydrolytic deamination to uracil and ammonia. Cytosine deaminases have been found in prokaryotes and lower eukaryotes, for example, the fungi Cryptococcus neoformans, Candida albicans, Torulopsis glabrata, Sporothrix schenckii, Aspergillus, Cladosporium, and Phialophora (J. E. Bennett, Chapter 50: Antifungal Agents, in Goodman and Gilman's the Pharmacological Basis of Therapeutics 8th ed., A. G. Gilman, ed., Pergamon Press, New York, 1990) and the bacteria Escherichia coli and Salmonella typhimurium (L. Andersen, et al., Archives of Microbiology, 152; 115-118, 1989 ). In these microorganisms the genetically encoded enzyme serves the same purpose: to help provide uracil from cytosine for nucleic acid synthesis. The E. coli enzyme and gene are representative of the group. Cytosine deaminase appears to be absent in higher eukaryotes, both in mammals as well as in plants. (Koechlin et al., Biochem Phannacol. 15, 435-446 (1966)); Ross, C., Plant Physiol. 40, 65-73 (1965)). Cytosine deaminase deaminates the innocuous fluorocytosine into fluorouracil, a highly toxic compound when efficiently converted to 5-fluoro-UMP. Thus, fusion proteins in accordance with the invention containing a sequence encoding cytosine deaminase and a mammalian DHFR can be used to provide localized toxicity upon administration of 5-fluorocytosine.

Other proteins within this class include varicella zoster TK (VZV-TK) gene. VZV-TK is able to covert the pro-drugs gancyclovir, acyclovir, 1-(2-deoxy-2-fluoro-.beta.-D-arabinofuranosyl)-5-iodouracil (FIAU) and 6-methoxypurine arabinoside into toxic substances. (See, U.S. Pat. No. 5,631,236 which is incorporated herein by reference).

Another class of proteins which may be incorporated as the therapeutic protein in the invention are products of a proapoptotic gene such as Bax, apoptin and Fas. The sequence of Bax is provided for mouse at GenBanK Accession No. NM_{—} 007527 (Seq. ID No. 8), for rat at GenBank Accession No. AB046392 (Seq. ID No. 9) and for human at GenBank Accession No. AX057142 (Seq. ID No. 10). Pas Protein sequences are known from GenBank Accession No. AAC 50124 (Seq. ID No. 11). Apoptin sequences are known from GenBank Accession No. NP_{—}056774 (Seq. ID No. 12). Increased expression of these proteins under control of the DHFR part of the fusion results in killing or suppression of transfected tumor cells by sensitizing them to chemotherapeutic drugs.

The therapeutic protein may also be a product of a tumor suppressor gene. Specific tumor suppressor genes include p53, p21, p27, p16 and p14. Increased expression of these proteins under control of the DHFR part of the fusion results in killing or suppression of transfected tumor cells by sensitizing them to chemotherapeutic drugs.

The therapeutic protein may also be an immunostimulatory molecule, such as an interleukin, macrophage stimulating factors, and interferons. Specific non-limiting examples of immunostimulatory molecules include IL-2, IL-12, GMCSF. Enhanced expression of such immunostimulatory molecules boosts the immune system so that T-cell mediated cell killing is augmented.

The therapeutic protein can also be a functional proteins useful in gene therapy. For example, DHFR-beta-globin can be used to provide an inducible source of a wild-type or enhanced beta-globin protein to replace a defective protein. Similarly, where the functional protein is one that needs some measure of regulation (for example insulin in response to glucose levels), expression can be regulated by administering antifolate in response to measured levels of a metabolite (i.e., glucose in the case of insulin).

In addition to the therapeutic proteins described above, the cDNA may also encode a reporter protein, such as green fluorescent protein, to facilitate monitoring of the extent and distribution of protein expression. The sequence of enhanced green fluorsecent protein (EGFP) is known from GenBank Accession No. L29345 (Seq. ID No. 13). The combination of DHFR and a reporter protein alone has utility as a diagnostic tool.

The cDNA molecules of the invention are made using techniques conventional for preparation of cDNA molecules encoding fusion proteins. In general, this involves amplification or cloning of a cDNA sequence encoding the desired parts of the ultimate fusion protein with matched restriction sites flanking each portion. Site-specific mutatgenesis can be used to introduce mutations, and to introduce restriction sites near or at the ends of amplification products. The cDNA sequence is then assembled into a corresponding restriction site in a vector of choice, cloned and selected. In the examples below, the DHFR is placed upstream of the therapeutic protein, and this is effective to allow DHFR inhibition to control expression of the fusion, but the invention is not limited to this orientation. The cDNA may be in the form of an expression vector including promoters appropriate to the subject to be treated. For example, expression of the fusion proteins in mammalian subjects, including humans, may be achieved using either strong viral promoters or tissue specific promoters. If desired, the cDNA may include an IRES to produce separate proteins, so that function in the expressed fusion would not be an issue. It should be noted, however, that antifolate-mediated translational upregulation may not be as effective when an IRES is present, as compared to the fusion protein without the IRES.

Because the cDNA molecules of the invention encode a new class of fusion proteins in which the therapeutic protein component can be essentially anything for which it is desirable to be able to control expression levels, they can be used in for a wide variety of applications. The resulting fusion proteins also constitute an aspect of the present invention. Such fusion proteins, in accordance with the invention comprise a DHFR portion and a therapeutic protein as described above. The term “fusion protein” means that this combination of a DHFR and a therapeutic protein are an artificial construct, and not a naturally occurring protein.

The cDNA molecules of the invention can be used to treat a wide variety of cancer types, including without limitation colorectal cancer, liver cancer, pancreatic cancer, lymphomas, lung cancer, prostate cancer and breast cancer using suicide genes as the therapeutic gene.

The cDNA molecules of the invention are administered to a mammalian subject to provide enhanced delivery of the therapeutic protein. The mammalian subject may be an animal, or a human. The cDNA can be administered as naked DNA or in a carrier such as a liposome or lipid particle, or in a viral construct, e.g,. retorvirus, adenovirus etc. Administration may be by intravenous, intramuscular or subcutaneous injection. In one specific embodiment of the invention which can be used for example in treatment of liver cancer, the cDNA is administered using the hepatic artery infusion (HAI) system now in use for delivery of fluorodeoxyurideine (FuDR) in liver metastasis of colorectal cancer. Ron and Kemany, Semin. Oncol. 26(5): 524-535 (1999).

The cDNA is administered in an amount sufficient to provide expression in target cells or tissues at levels sufficient to produce a therapeutic benefit. In the case of cDNA which encodes a fusion protein containing a suicide gene such as HSV-TK, the amount administered of cDNA is suitably enough to transduce 10 to 20% or more of the target tumor cells and associated neovasculature. In the case of other types of fusion proteins, the amount of cDNA administered and the dosage schedule will depend on the desired levels of the therapeutic protein, the efficiency of expression and the stability of transduction. The appropriate level can be determined for any given fusion through routine testing.

Once the cDNA molecule has been administered, expression can be stimulated through subsequent administration of compounds such as antifolates which overcome the repression of DHFR expression and folates such as dihydrofolate. Specific non-limiting examples of appropriate antifolates include methotrexate, trimetrexate and aminopterins, such as 10 propargyl-10-deazaaminopterin. (See U.S. Pat. No. 6,028,071 which is incorporated herein by reference). The antifolate can be administered by any of the routes mentioned above for the cDNA, and is preferably administered by the same route as the cDNA in a given therapy to facilitate delivery to the same sites. The antifolate may be administered in a single or in multiple periodic bolus administrations, or as a continuous administration over a period of time. The amount administered is bounded at the upper limit by toxicity issues. For example, in the case of TMTX, the known toxicity levels indicate that an appropriate therapeutic dosage would be in the range of from 0.1 to 10 μM. Variable amounts less than toxic levels may be supplied to result in variable amounts of expression of the therapeutic protein. Leucovorin may be coadministered to reduce toxic side effects.

### EXAMPLE 1

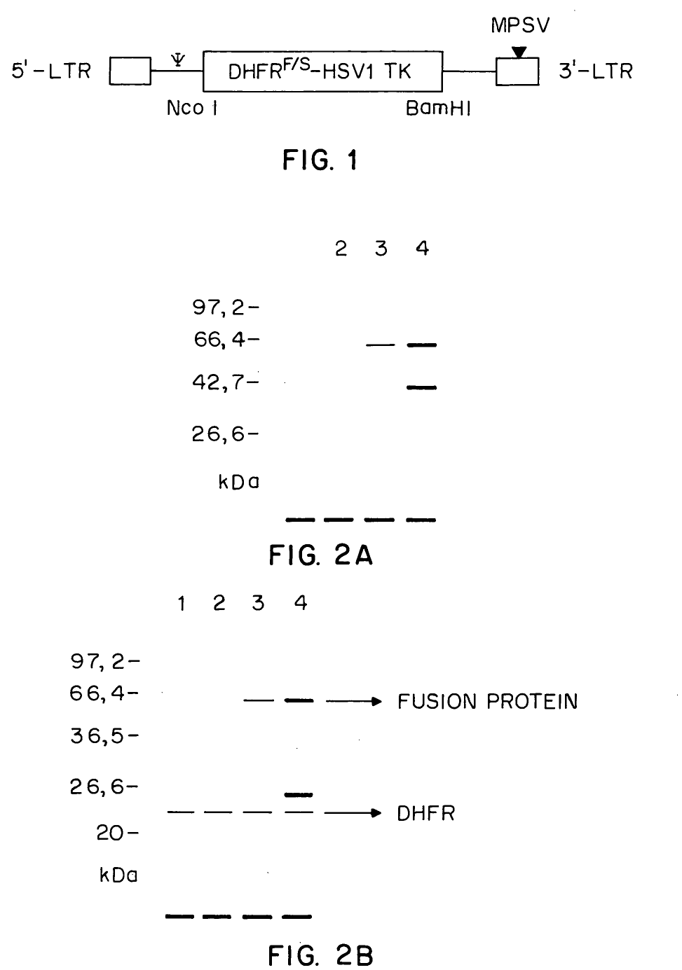

A fusion cDNA was constructed as illustrated in FIG. 1. As shown, this cDNA contains a sequence encoding a double mutant (Phe22 Ser31) of human DHFR and HSV-thymidine kinase in an SPG-based retroviral vector as described in Riviere et al., Proc. Nat'l Acad. Sci. (USA) 92: 6733-6737 (1995). The coding sequence for the double mutant DHFR was as set forth in Ercikan-Abali et al., Cancer Res. 56: 4142-445 (1996). A published coding sequences for HSV-TK (accession no. AB009258, (Seq. ID No. 14)) was used.

Retrovirus-producing cell-lines were generated by transfection of DHFR-HSV1-TK plasmid in parental GP+envAM12 cells. Transfection was performned three times at cell densities of 30%, 50% and 75% using Superfect (Qiagen, Chatsworth, Calif.) as described by the manufacturer. Clones of the retrovirus were selected in 150 nM TMTX. Viral titers were determined against NIH 3T3 cells. Retrovirus-producing AM12V cells were grown in medium without antifolate to a density of 60-80%; the day before infection, the mdeium was changed. Viral transduction was performed by infecting colon cancer cells plated 48 hours before infection in 10 cm dishes. Four single virus exporsures with the desired multiplicity of infection (moi), each of 6 hour duration, were carried out. For each single exposure, fresh AM12V supernatant was filtered through a 0.45 μm cellulose acetate filter and supplemented with polybrene (8 μg/ml). The 2-hour viral transduction was performedin 6 well plates.

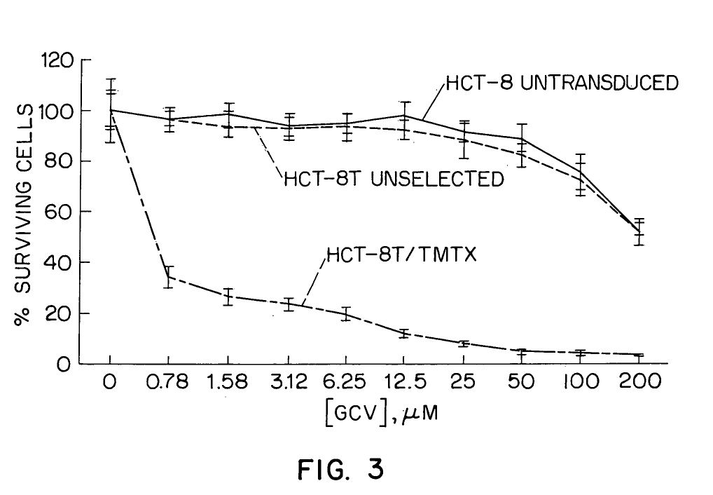

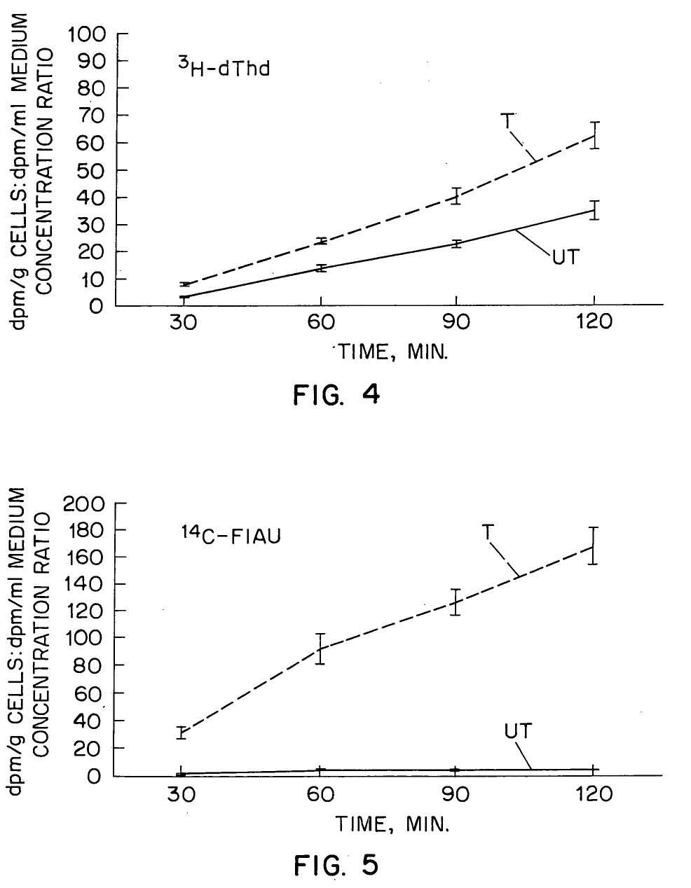

The transduced cells were treated with varying amounts of TMTX (10, 100 and 1000 μM) and levels of fusion protein were detected by Western blotting using either an anti-HSV-TK antibody or an anti-human DHFR antibody. As shown in FIG. 2, upregulation of fusion protein expression was observed. These transduced cells showed increased sensitivity to GCV as compared to unselected cells or untransduced cells (FIG. 3).

### EXAMPLE 2

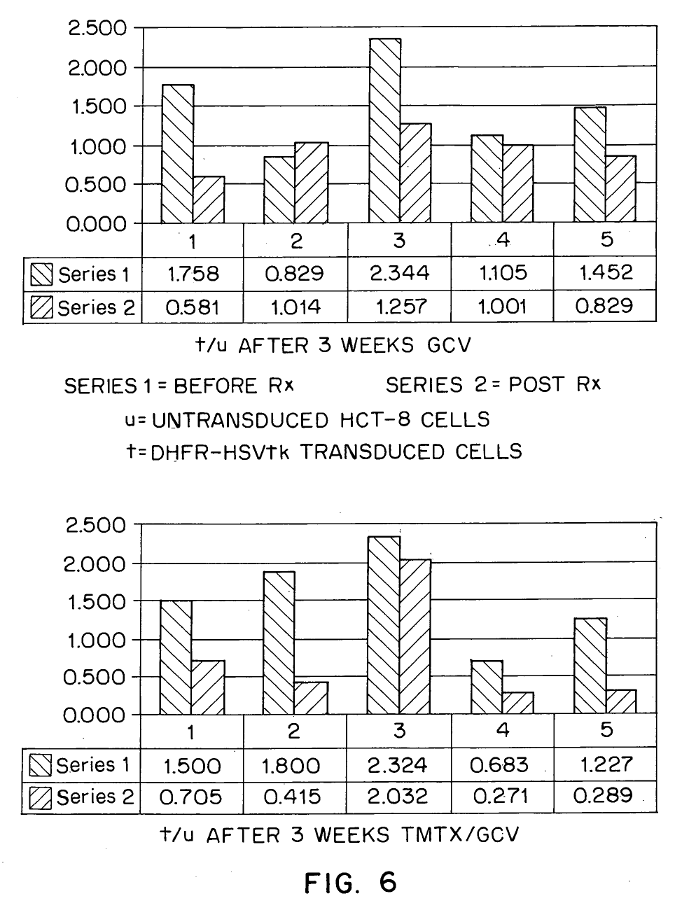

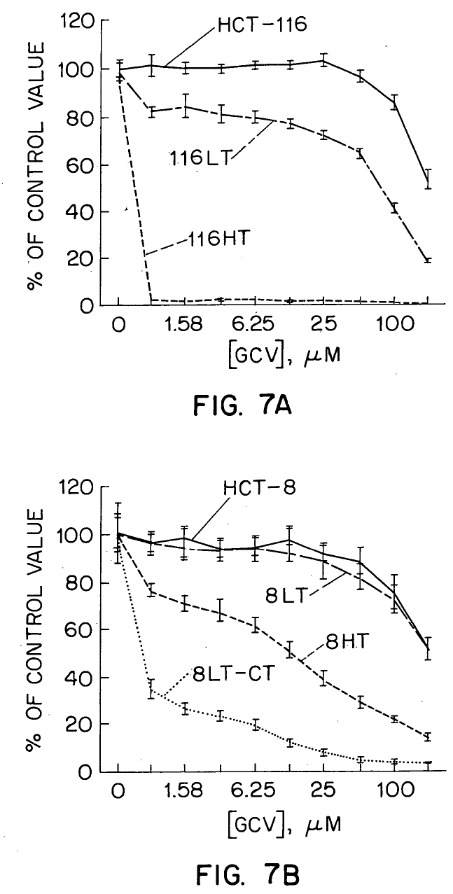

The transduced HCT-8 cells of accumulated more ¹⁴C-FIAU than untransduced cells (where accumulation was not detectable), indicating HSV-TK activity. ³H-thymidine incorporation was comparable. (FIGS. 4 and 5) PET scanning was used for demonstration of in vivo upregulation of the fusion gene scanning. Nude rats bearing human colon tumor cells (HCT-8 and C-85) were transduced ex vivo with the SFG-DHFR/HSVTK vector (FIG. 1) and subsequently treated by intraperitoneal injeciton with either acute (50 mg/kg at 10 am and 6 pm on day before imaging) or chronic doses (10 mg/kg 3 times per week for 3 weeks before imaging (total of 9 doses)) of TMTX. A radiolabeled substrate of HSV-TK (¹²⁴I-FIAU, 2-fluoro-1β-D-arabino-furanosyl-5-iodo-uracil) was used to image tumors in vivo (250-400 μCi per animal via the penile vein). Tumors that had been transduced with the fusion cDNA vector showed increased image intensity by PET scanning as opposed to untransformed tumors. Furthermore, better anti-tumor response of transduced tumors was observed using TMTX/GCV combination therapy than using either TMTX or GMCV alone. (FIG. 6).

### EXAMPLE 3

Three colorectal cancer cells lines (HCT-8, HCT-116 and a cell line, C85, recently established from a patient with liver metastasis) were transduced with the SFG-DHFR/HSV-TK fusion vector of Example 1. Transduction was verified by PCR using transgene specific primers and elevation of DHFR-HSVTK mRNA and protein expression. After treatment of the transduced cells with several cycles of nine day exposures to increasing concentration of TMTX, a stable increase in mRNA and protein levels of the fusion protein was seen. Furthermore, these cells exhibits a 250-fold increase in sensitivity to GCV and significantly increased accumulation of ¹⁴C-FIAU as compared to non-transduced cells. A transient increase in fusion protein levels without an increase in mRNA was observed following treatment of the cells with high concentrations (1000 μM) of TMTX, methotrexate or dihydrofolate.

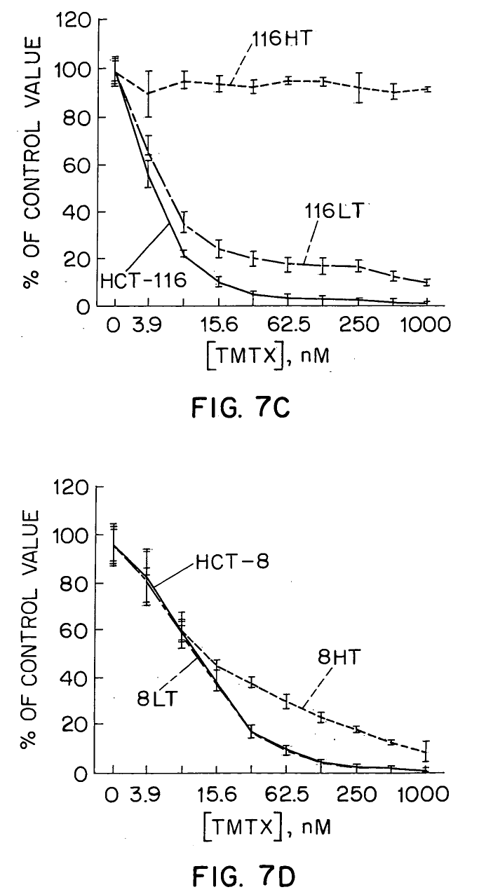

FIGS. 7A-D show GCV sensitivty and TMTX resisatnce in parental, tranduced and TMTX exposed colon cancer cells of the HCT-116 and HCT-8 cells lines. Bulk transduction of HCT-116 and HCT-8 was carried out with an moi of 30 for 24 hours or an moi of 2.5 for 2 hours to produce cell lines 116HT and 116LT, and 8HT and 8LT, respectively. Successful gene transfer and transgene expression was confirmed by PCR and Western blotting. The percentage of infected cells was determined by colony formation assay using the GCV sensitivity of DHFR-HSV1-TK-expressing cells to be as follows: 116HT, very high (no colony formation); 116LT, 80%; 8HT, 53%. Infection of 8LT was not detectable with the assay used.

GCV amd TMTX cytotoxicities were measured on the transduced cells. 116LT cells exhibited a moderate and 116HT a higher GCV and TMTX resistance as compared with parental cell lines. (FIGS. 7A and C). This finding correlates with observed levels of fusion protein expression in these cells. In a similar way, detected fusion protein expression correlates with the drug cytotoxicity in transduced HCT-8 cells: 8HT cells, but not in 8LT cells showed GCV sensitivity and TMTX resistance ((FIGS. 7B and D).

When HCT-8 cells were treated chronically with increasing concentrations of TMTX, a 250-fold increase in GCV sensitivity was measured (FIG. 7B, line 8LT-CT), accompanied by an increased gene copy number and fusion protein expression. This induction of expression of the DHFR-HSV1 TK fusion protein was shown to be dose-dependent with both MTX and TMTX over concentrations ranges of 10⁻² to 1 μM. Induction was also observed with the DHFR substrate, dihydrofolate, at a concentration of 50 μM. In contrast, treatment of cells with etoposide, a topoisimerase II inhibitor, did not cause induction of the fusion protein.

### EXAMPLE 4

Rats bearing flank tumors were treated with 10 mg/kg of TMTX for three days (once daily), or with a single high dose of 100 mg/kg. The levels of DHFR-HSV1 TK fusion protein in the tumor tissue of antifolate treated rats, as well as in water-treated controls were analyzed. All antifolate treated animals showed elevated levels of the fusion protein, ranging from 1.5 to 4-fold relative to the controls. The mean increase was at least 2-fold.

In a second set of experiments, tumors derived from 8LT-CT and parental HCT-8 cells were evaluated by in vivo imaging. Before the imaging, each animal received three cycles of 10 mg/kg of TMTX, three times daily for three days per cycle. Using PET and the tracer ¹²⁴I-FIAUan increase in tumor-signal intensity in TMTX-treated rats was observed. Measurements of ¹²⁴I-FIAU uptake (% dose per g) from 7 treated and 7 control rats showed a 2.6 fold-higher ¹²⁴I- FIAU accumulation in transduced tumor tissue following TMTX treatment as compared with untreated controls.

### EXAMPLE 5

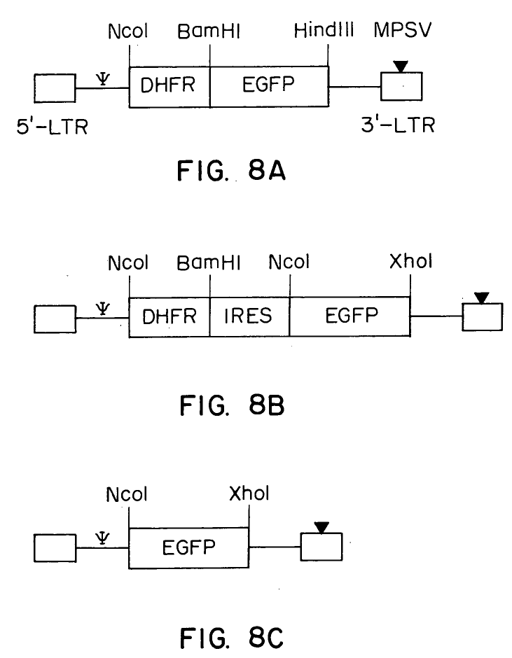

SFG-based retroviral vectors were prepared encoding EGFP, DHFR-EFGP and DHFR and EGFP separated by an IRES and shown in FIG. 8. The sequence for EGFP is known from published sequences (accession no. for cloning vector pEGFP-1 is U55761, (Seq. ID No. 15)) The procedures for introduction of the IRES were as described in Frebourg et al., Cancer Res. 54: 876-881 (1994).

Transfections of parental GP+envAM12 cells (Markowitz et al., Virology 167:400-405 (1988)) with EGFP-containing SFG-plasmids was carried out using DOTAP transfecting reagent (N-{1-(2,3-dioleyloxy)propyl]-N,N,N,-trimethylammonium methyl sulfate, Roche Molecular Biochemicals). Cells transfected with EGFP-expressing vectors were cultrued for at least 12 days and subsequently sorted for EGFP-positive cells using an FACS Vantage SE cell sorter (Becton Dickinson). For fluorescence microscopy, these cells were grown in T25 flasks. Confluent cells were cultured in fresh media with or without TMTX. Fluorescent images of randomly selected areas of the monolayer were taken with a Zeiss Axlovert S100 microscope.

In cells transfected with the DHFR-EGFP fusion vector, cellular expression of the fusion protein was observed. This expression was increased upon treatment with 1 μM of TMTX, and this increase was durable at 24 and 48 hours after treatrment. Cells transfected with plasmids expressing DHFR and EGFP separated by an IRES, or just EGFP both resulted in expression of EGFP at higher levels than cells transfected with DHFR-EGFP fusion vectors, but this was not increased by the addition of TMTX.

What is claimed is:

1. A cDNA molecule encoding a fusion protein that comprises mammalian DHFR and a therapeutic protein.

2. The cDNA of claim 1, wherein the fusion protein comprises a wild-type mammalian DHFR.

3. The cDNA of claim 2, wherein the wild-type mammalian DHFR is rat, mouse, dog, monkey or human DHFR.

4. The cDNA of claim 1, wherein the fusion protein comprises a mutant form of DHFR.

5. The cDNA of claim 4, wherein the fusion protein comprises a mutant form of human DHFR.

6. The cDNA of claim 5, wherein the mutant form of human DHFR has increased resistance to methotrexate.

7. The cDNA of claim 4, wherein the mutant form of human DHFR differs from wild-type DHFR as a result of one or more mutations, including at least one mutation at an amino acid corresponding to amino acid 15, 22, 31 or 34 of the wild-type sequence.

8. The eDNA of claim 7, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

9. The cDNA of claim 1, wherein the therapeutic protein is a protein that enhances toxicity of an administered drug.

10. The cDNA of claim 9, wherein the therapeutic protein is a mutant or wild-type form of herpes simplex virus thymidine kinase.

11. The cDNA of claim 10, wherein the fusion protein comprises a wild-type mammalian DHFR.

12. The cDNA of claim 11, wherein the wild-type mammalian DHFR is rat, mouse, dog, monkey or human DHFR.

13. The cONA of claim 10, wherein the fusion protein comprises a mutant form of DHFR.

14. The cDNA of claim 13, wherein the fusion protein comprises a mutant form of human DHFR.

15. The cDNA of claim 14, wherein the mutant form of human DHFR has increased resistance to methotrexate.

16. The cDNA of claim 13, wherein the mutant form of human DHFR differs from wild-type DHFR as a result of one or more mutations, including at least one mutation at an amino acid corresponding to amino acid 15, 22, 31 or 34 of the wild-type sequence.

17. The cDNA of claim 16, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

18. The cDNA of claim 9, wherein the therapeutic protein is cytosine deaminase.

19. The cDNA of claim 1, wherein the therapeutic protein is a product of a pro-apoptotic gene.

20. The cDNA of claim 19, wherein the fusion protein comprises a wild-type mammalian DHFR.

21. The cDNA of claim 19, wherein the fusion protein comprises a mutant form of DHFR.

22. The cDNA of claim 21, wherein the fusion protein comprises a mutant form of human DHFR.

23. The cDNA of claim 22, wherein the mutant form of human DHFR has increased resistance to methotrexate.

24. The cDNA of claim 22, wherein the mutant form of human DHFR differs from wild-type DHIFR as a result of one or more mutations, including at least one mutation at an amino acid corresponding to amino acid 15, 22, 31 or 34 of the wild-type sequence.

25. The cDNA of claim 24, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

26. The cDNA of claim 1, wherein the therapeutic protein is a product of a tumor suppressor gene.

27. The cDNA of claim 19, wherein the fusion protein comprises a wild-type mammalian DHFR.

28. The cDNA of claim 27, wherein the fusion protein comprises a mutant form of DHFR.

29. The cDNA of claim 28, wherein the fusion protein comprises a mutant fonrm of human DHFR.

30. The cDNA of claim 29, wherein the mutant form of human DHFR has increased resistance to methotrexate.

31. The cDNA of claim 29, wherein the mutant form of human DHFR differs from wild-type DHFR as a result of one or more mutations, including at least one mutation at an amino acid corresponding to amino acid 15, 22, 31 or 34 of the wild-type sequence.

32. The cDNA of claim 31, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

33. The cDNA of claim 1, wherein the therapeutic protein is an immunostimulatory molecule.

34. The cDNA of claim 33, wherein the fusion protein comprises a wild-type mammalian DHFR.

35. The cDNA of claim 33, wherein the fusion protein comprises a mutant form of DHFR.

36. The cDNA of claim 35, wherein the fusion protein comprises a mutant form of human DHFR.

37. The cDNA of claim 36, wherein the mutant form of human DHFR has increased resistance to methotrexate.

38. The cDNA of claim 36, wherein the mutant form of human DHFR differs from wild-type DHFR as a result of one or more mutations, including at least one mutation at anamino acid corresponding to amino acid 15, 22, 31 or 34 of the wild-type sequence.

39. The cDNA of claim 38, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

40. The cDNA of claim 1, wherein the therapeutic protein is a functional protein useful in gene therapy.

41. The cDNA of claim 40, wherein the therapeutic protein is a wild-type or enhanced beta-globin protein.

42. The cDNA of claim 1, further comprising a region encoding a reporter protein.

43. The cDNA of claim 42, wherein the reporter protein is green fluorescent protein.

44. A fusion protein that comprises mammalian DHFR and a therapeutic protein.

45. The fusion protein of claim 44, wherein the fusion protein comprises a wild-type mammalian DHFR.

46. The fusion protein claim 46, wherein the wild-type mammalian DHFR is rat, mouse, dog, monkey or human DHFR.

47. The fusion protein of claim 44, wherein the fusion protein comprises a mutant form of DHFR.

48. The fusion protein of claim 47, wherein the fusion protein comprises a mutant form of human DHFR.

49. The fusion protein of claim 48, wherein the mutant form of human DHFR has increased resistance to methotrexate.

50. The fusion protein of claim 47, wherein the mutant form of human DHFR differs from wild-type DHFR as a result of one or more mutations, including at least one mutation at an amino acid corresponding to amino acid 15, 22, 31 or 34 of the wild-type sequence.

51. The fusion protein of claim 50, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

52. The fusion protein of claim 44, wherein the therapeutic protein is a protein that enhances toxicity of an administered drug.

53. The fusion protein of claim 52, wherein the therapeutic protein is a mutant or wild-type form of herpes simplex virus thymidine kinase.

54. The fusion protein of claim 52, wherein the therapeutic protein is cytosine deaminase.

55. The fusion protein of claim 44, wherein the therapeutic protein is a product of a pro-apoptotic gene.

56. The fusion protein of claim 44, wherein the therapeutic protein is a product of a tumor suppressor gene.

57. The fusion protein of claim 44, wherein the therapeutic protein is an immunostimulatory molecule.

58. The fusion protein of claim 44, wherein the therapeutic protein is a functional protein useful in gene therapy.

59. The fusion protein of claim 58, wherein the therapeutic protein is a wild-type or enhanced beta-globin protein.

60. The fusion protein of claim 44, further comprising a reporter protein.

61. The fusion protein of claim 60, wherein the reporter protein is green fluorescent protein.

62. A method for providing enhanced delivery of a therapeutic protein to a mammalian subject comprising the step of administering to the mammalian subject a cDNA molecule encoding a fusion protein that comprises mammalian DHFR and a therapeutic protein such that the cDNA molecule is expressed

63. The method of claim 62, wherein the fusion protein comprises a wild-type mammalian DHFR.

64. The method of claim 63, wherein the wild-type mammalian DHFR is rat, mouse, dog, monkey or human DHFR.

65. The method of claim 62, wherein the fusion protein comprises a mutant form of DHFR.

66. The method of claim 65, wherein the fusion protein comprises a mutant form of human DHFR.

67. The method of claim 66, wherein the mutant form of human DHFR has increased resistance to methotrexate.

68. The method of claim 66, wherein the mutant form of human DHFR differs from wild-type DHFR as a result of one or more mutations, including at least one mutation at an amino acid corresponding to amino acid 15, 22, 31 or 34 of the wild-type sequence.

69. The method of claim 68, wherein the mutant form of human DHFR differs from wild-type human DHFR as a result of a set of mutations comprising a mutation at the amino acid corresponding to amino acid 22 and a mutation at the amino acid corresponding to amino acid 31 of the wild-type sequence.

70. The method of claim 62, wherein the therapeutic protein is a protein that enhances toxicity of an administered drug.

71. The method of claim 70, wherein the therapeutic protein is a mutant or wild-type form of herpes simplex virus thymidine kinase.

72. The method of claim 70, wherein the therapeutic protein is cytosine deaminase.

73. The method of claim 62, wherein the therapeutic protein is a product of a pro-apoptotic gene.

74. The method of claim 62, wherein the therapeutic protein is a product of a tumor suppressor gene.

75. The method of claim 62, wherein the therapeutic protein is an immunostimulatory molecule.

76. The method of claim 62, wherein the therapeutic protein is a functional protein useful in gene therapy.

77. The method of claim 76, wherein the therapeutic protein is a wild-type or enhanced beta-globin protein.

78. The method of claim 62, wherein the fusion protein further comprises a reporter protein.

79. The method of claim 78, wherein the reporter protein is green fluorescent protein.
